(19) United States
(12) Patent Application Publication
(10) Pub. No.: US 2007/0027533 A1
(43) Pub. Date: Feb. 1, 2007

(54) Cardiac valve annulus restraining device

(75) Inventor: Nareak Douk, Lowell, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/193,674

(22) Filed: Jul. 28, 2005

(51) Int. Cl. A61F 2/24

(52) U.S. Cl. 623/2.11; 623/2.37

(57) ABSTRACT

A catheter based system for treating mitral valve regurgitation includes a restraining device having a flexible member, a plurality of movable anchor members attached to the outer surface of the flexible member, and an adjustment filament attached to the ends of the flexible member. One embodiment of the invention includes a method for attaching a flexible restraining device to the annulus of a mitral valve, and adjusting the length of the adjustment filament attached to the flexible member of the restraining device, thereby reshaping the mitral valve annulus so that the anterior and posterior leaflets of the mitral valve close during ventricular contraction.

### TECHNICAL FIELD

This invention relates generally to medical devices for treating mitral valve regurgitation, and particularly to a cardiac valve annulus restraining system and method of using the same.

### BACKGROUND OF THE INVENTION

Heart valves, such as the mitral, tricuspid, aortic and pulmonic valves, are sometimes damaged by disease or by aging, resulting in problems with the proper functioning of the valve. Heart valve problems take one of two forms: stenosis, in which a valve does not open completely or the opening is too small, resulting in restricted blood flow; or insufficiency, in which blood leaks backward across a valve when it should be closed. Valve replacement may be required in severe cases to restore cardiac function. In common practice, repair or replacement requires open-heart surgery with its attendant risks, expense, and extended recovery time. Open-heart surgery also requires cardiopulmonary bypass with risk of thrombosis, stroke, and infarction.

Mitral valve insufficiency results from various types of cardiac disease. Any one or more of the mitral valve structures, i.e., the anterior or posterior leaflets, the chordae, the papillary muscles or the annulus may be compromised by damage from disease or injury, causing the mitral valve insufficiency. In cases where there is mitral valve insufficiency, there is some degree of annular dilatation resulting in mitral valve regurgitation. Mitral valve regurgitation occurs as the result of the leaflets being moved away from each other by the dilated annulus. Thus, without correction, the mitral valve insufficiency may lead to disease progression and/or further enlargement and worsening of the insufficiency. In some instances, correction of the regurgitation may not require repair of the valve leaflets themselves, but simply a reduction in the size of the annulus. A variety of techniques have been used to reduce the diameter of the mitral annulus and eliminate or reduce valvular regurgitation in patients with incompetent valves.

Current surgical procedures to correct mitral regurgitation in humans include a number of mitral valve replacement and repair techniques. Valve replacement can be performed through open-heart surgery, open chest surgery, or percutaneously. The native valve is removed and replaced with a prosthetic valve, or a prosthetic valve is placed over the native valve. The valve replacement may be a mechanical or biological valve prosthesis. The open chest and percutaneous procedures avoid opening the heart and cardiopulmonary bypass. However, the valve replacement may result in a number of complications including a risk of endocarditis. Additionally, mechanical valve replacement requires subsequent anticoagulation treatment to prevent thromboembolisms.

As an alternative to valve replacement, various valve repair techniques have been used including quadrangular segmental resection of a diseased posterior leaflet, transposition of posterior leaflet chordae to the anterior leaflet, valvuloplasty with plication and direct suturing of the native valve, substitution, reattachment or shortening of chordae tendinae, and annuloplasty in which the effective size of the valve annulus is contracted by attaching a prosthetic annuloplasty ring to the endocardial surface of the heart around the valve annulus. The annuloplasty techniques may be used in conjunction with other repair techniques. Annuloplasty rings are sometimes sutured along the posterior mitral leaflet adjacent to the mitral annulus in the left atrium. The rings either partially or completely encircle the valve, and may be rigid, or flexible but non-elastic. All of these procedures require cardiopulmonary bypass, though some less, or minimally invasive techniques for valve repair and replacement are being developed.

Although mitral valve repair and replacement can successfully treat many patients with mitral valve insufficiency, techniques currently in use are attended by significant morbity and mortality. Most valve repair and replacement procedures require a thoracotomy, to gain access to the patient's thoracic cavity. Surgical intervention within the heart frequently requires isolation of the heart and coronary blood vessels from the remainder of the arterial system and arrest of cardiac function. Open chest techniques with large sternum openings are used. Those patients undergoing such techniques often have scarring retraction, tears or fusion of valve leaflets, as well as disorders of the subvalvular apparatus.

Recently, other surgical procedures have been provided to reduce the mitral valve annulus using a less invasive surgical technique. According to this method, a prosthesis is transvenously advanced into the coronary sinus and deployed within the coronary sinus to reduce the diameter of the mitral valve annulus. The prosthesis then undergoes a change within the coronary sinus that causes it to assume a reduced radius of curvature, and as a result, to reduce the circumference of the mitral valve annulus. This may be accomplished in an open procedure or by percutaneously accessing the venous system by one of the internal jugular, subclavian or femoral veins.

While the coronary sinus implant provides a less invasive treatment alternative, the placement of the prosthesis within the coronary sinus may be problematic for a number of reasons. Sometimes the coronary sinus is not accessible. The coronary sinus on a particular individual may not wrap around the heart far enough to allow enough encircling of the mitral valve. Also, leaving a device in the coronary sinus may result in the formation of thrombus, which may break off and pass into the right atrium, right ventricle and ultimately the lungs causing a pulmonary embolism. Another disadvantage is that the coronary sinus is sometimes used for placement of a pacing lead, which may be precluded with the placement of the prosthesis in the coronary sinus.

Therefore, it would be desirable to provide a method and device for reducing cardiac valve regurgitation that use minimally invasive surgical techniques, and would overcome the limitations and disadvantages inherent in the devices described above.

### SUMMARY OF THE INVENTION

One aspect of the present invention provides a system for treating mitral valve regurgitation comprising a delivery catheter and a flexible restraining device. The restraining device comprises a flexible member having a plurality of anchor members, and adjustment members attached to the end portions of the flexible member. The restraining device has an elongated essentially linear configuration for catheter delivery to a location adjacent a mitral valve annulus and an arcuate configuration, which it assumes after it is deployed from a delivery catheter. When the restraining device is deployed from the delivery catheter, the barbs move from a delivery position to a deployment position and engage with the mitral valve annulus. Using the adjustment member, the radius of the flexible restraining members is adjusted causing a corresponding change in the shape of the mitral valve annulus.

Another aspect of the invention provides a device for treating mitral valve regurgitation. The device includes a flexible restraining member having a plurality of anchor members extending from the flexible restraining member and at least one adjustment member attached to the end portions of the flexible restraining member. When the device is deployed from a delivery catheter, the barbs move from a delivery position to a deployment position and engage the annulus of the mitral valve. The radius of the flexible restraining member can then be adjusted via the adjustment members, causing the shape of the mitral valve annulus to change, and regurgitation to be reduced.

Another aspect of the invention provides a method for treating mitral valve regurgitation. The method comprises using a catheter to deliver a flexible restraining device having shape-memory barbs adjacent to a location adjacent a mitral valve, deploying the flexible restraining device from the distal tip of the catheter, and moving the barbs from a delivery position to a deployment position in response to the deployment of the flexible device from the catheter. The method further comprises positioning the flexible device against the annulus of the mitral valve, inserting the anchor members into the annulus, and altering the radius of an arcuate portion of the flexible member. The mitral valve annulus is reshaped in response to the altering of the radius of the arcuate portion of the flexible member.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a cross sectional schematic view of a heart showing the location of the mitral valve;

FIG. 2 is a view of a flexible restraining device having a flexible member and movable barbs in a deployment position, in accordance with the present invention;

FIG. 3 is a view of a flexible restraining device having movable barbs in a delivery position, in accordance with the present invention;

FIG. 4 is a view of a flexible restraining device in an elongated delivery configuration, in accordance with the present invention;

FIG. 5 is a side view of a flexible restraining device in an elongated configuration inside the distal portion of a delivery catheter, in accordance with one aspect of the invention;

FIG. 6 is a schematic view illustrating the placement of the flexible restraining device adjacent to the mitral valve, in accordance with one aspect of the invention;

FIG. 7 is a schematic view of a delivery system for the flexible restraining devices, in accordance with one aspect of the invention;

FIG. 8 is a view of a wireform in an elongated configuration, in accordance with one aspect of the invention;

FIG. 9 is a side view of a delivery catheter for delivering a wireform adjacent to the mitral valve, in accordance with one aspect of the invention;

FIG. 10 is a flow diagram of a method of treating mitral valve regurgitation in accordance with one aspect of the invention.

### DETAILED DESCRIPTION

Throughout this specification, like numbers refer to like structures.

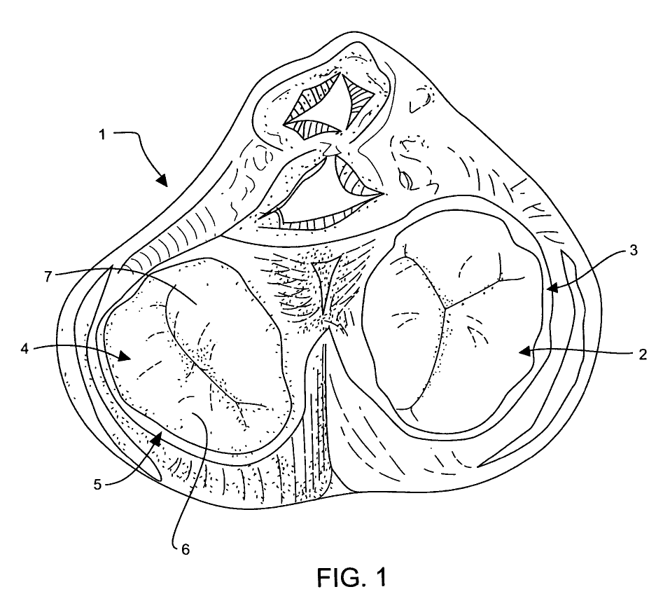

Referring to the drawings, FIG. 1 shows a cross-sectional view of heart 1 having tricuspid valve 2 and tricuspid valve annulus 3. Mitral valve 4 is adjacent mitral valve annulus 5. Mitral valve 4 is a bicuspid valve having anterior cusp 7 and posterior cusp 6. Anterior cusp 7 and posterior cusp 6 are often referred to, respectively, as the anterior and posterior leaflets.

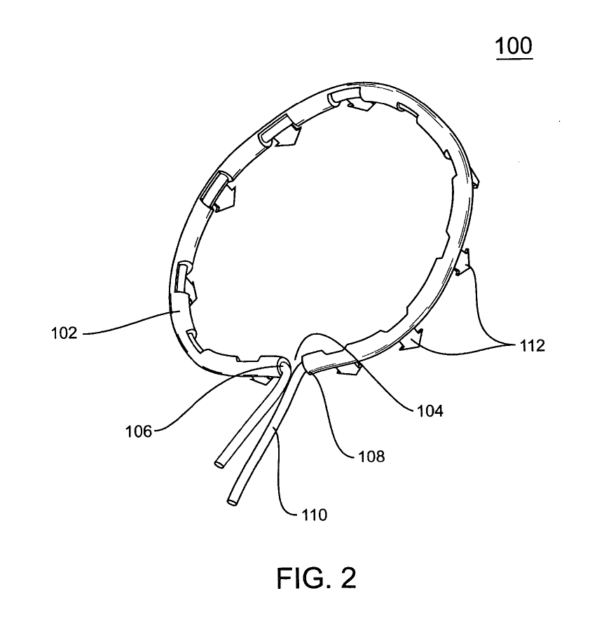

FIG. 2 portrays a flexible restraining device 100 for treating mitral valve regurgitation. Restraining device 100 includes a flexible member 102 that is depicted in the figure in an arcuate shape that the member will assume upon delivery to a location adjacent a mitral valve. Flexible member 102 is made of a flexible, biocompatible material that has “shape memory” so that flexible member 102 can be extended into an elongated configuration and inserted into a delivery catheter, but will assume a curved shape and dimensions when deployed adjacent to the mitral valve annulus. In one embodiment of the invention, flexible member 102 comprises nitinol, a biocompatible material that gives flexible member 102 the needed flexibility and shape memory. Fabrication of flexible member 102 may include chemical machining, forming or heat setting of nitinol. In addition, the surface of flexible member 102 should be hemocompatible, and cause minimal blood clotting or hemolysis when exposed to flowing blood. In one embodiment of the invention, flexible member 102 comprises a flexible, nitinol ring with a cover. In one embodiment, the cover is composed of a polyester fiber. Dacron®, polyester fiber (E.I. Du Pont De Nemours & Co., Inc.) is a material known in the art to have the necessary hemocompatible properties and may be used in the cardiovascular system.

The size and shape of flexible member 102 are selected to fit the configuration of the mitral valve annulus. In one embodiment of the invention, flexible member 102 is circular in shape except for a small gap 104.

Extending from each of ends 106 and 108 of flexible member 102 are flexible adjustment members 110. In one embodiment of the invention, the adjustment members 110 are firmly attached to ends 106 and 108 of flexible member 102 and comprise a filament, string, wire, cord or cable. In another embodiment of the invention, flexible member 102 comprises a hollow flexible tube and adjustment member 110 is a single wire extending through the interior lumen of flexible member 102, and protruding from ends 106 and 108 as shown in FIG. 2. In either embodiment, adjustment members 110 are used to draw ends 106 and 108 of flexible member 102 toward each other, and reshape restraining device 100 by reducing gap 104, and changing the radius of flexible member 102. The ends of adjustment wire 110 may then be twisted around each other to maintain gap 104 at a reduced size. In other embodiments in which adjustment member 110 comprises a filament or other highly flexible material, the ends of adjustment member 110 are drawn toward each other and knotted, or held in place with a locking assembly, such as a clamp lock, or any other appropriate device.

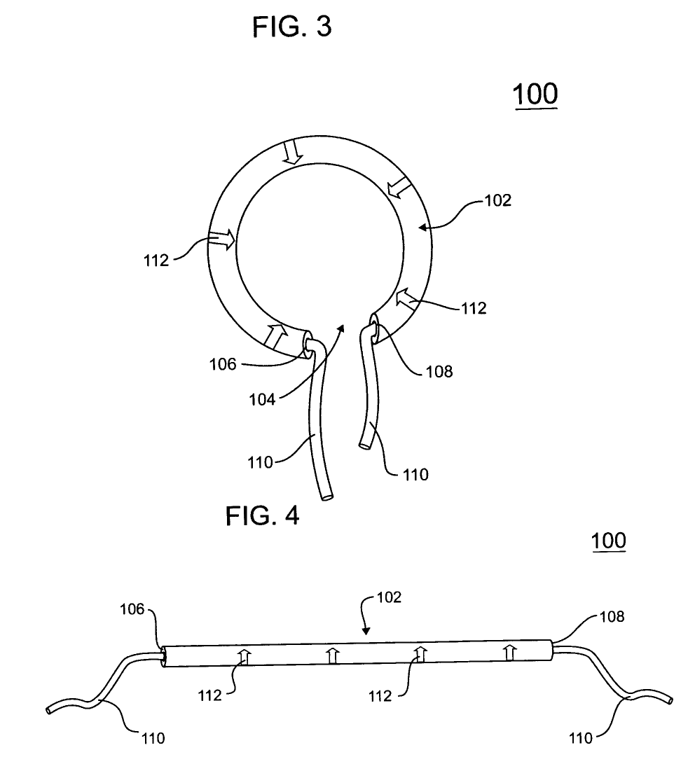

A plurality of anchor members, comprising barbs or prongs 112, are disposed about the exterior surface of flexible member 102, and are used to attach flexible member 102 to the mitral valve annulus. In one embodiment of the invention, anchor members 112 are formed by laser cutting the wall of flexible member 102 in such a manner as to create sharp pointed portions in a plurality of locations. These sharp pointed portions may then be shaped into anchoring barbs 112, and then manipulated so that they are oriented at an angle of 45-90 degrees in relation to the surface of flexible member 102, and heat set in this open position, as seen in FIG. 2. However, anchor members 112 are flexible, and may be pressed back into the planer surface of flexible member 102 to assume a closed position as shown in FIG. 3. In this closed position, anchor members 112 form part of the smooth exterior surface of flexible member 102, and facilitate delivery of device 100 via catheter.

Flexible member 102 can be transformed from its curved, nearly circular configuration (FIG. 2, 3) into an elongated, substantially linear configuration (FIG. 4). The two ends 106, 108 may be moved in opposite directions until device 100 is in an elongated, substantially linear configuration. Because flexible member 102 comprises a shape-memory material, such as nitinol, device 100 will spontaneously revert to an unconstrained, flexible or curved configuration (FIG. 2) when free to do so.

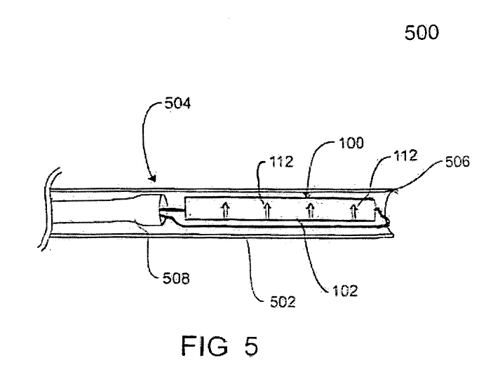

FIG. 5 is a side view of the distal portion of system 500 for treating mitral valve regurgitation using minimally invasive surgical techniques, in accordance with the present invention. Flexible restraining device 100 is contained within a sheath 502 forming a delivery chamber in the distal portion of delivery catheter 504. Delivery catheter 504 is flexible, and configured so that it can be inserted into the cardiovascular system of a patient. Such catheters are well known in the art and are, for example, between 5 and 12 French in diameter. Appropriate catheters are made of flexible biocompatible materials such as polyurethane, polyethylene, nylon and polytetrafluoroethylene (PTFE). In order to facilitate passage through the vascular system, distal sheath 502 may have greater lateral flexibility than the tubular body of catheter 504. In one embodiment of the invention, an inflatable balloon is attached to the distal portion of catheter 504, and connected by a lumen to a reservoir of liquid at the proximal end of catheter 504.

Flexible member 102 of restraining device 100 is opened to its elongated configuration, and anchor members 112 are in the closed, delivery position, forming a smooth exterior surface, as shown in FIG. 4. Restraining device 100 is then placed within the lumen of catheter 504 near catheter distal tip 506. Within the lumen of catheter 504, and proximal to restraining device 100 is a deployment device, such as delivery member 508. The delivery member 508 is made from a flexible material and it is used to deploy restraining device 100 by pushing it from catheter distal tip 506. In the depicted embodiment, the delivery member is a hollow member having an enlarged end portion that is adapted such that an end of the restraining device can fit therein during delivery and the be easily deployed therefrom. In the depicted embodiment, the adjustment members extending from the ends of the restraining device are routed into the delivery member during deployment of the restraining device. After restraining device 100 is deployed, the delivery member 508 may be withdrawn from catheter 504. In one embodiment of the invention, the interior surface of catheter 504 is coated with a lubricious material such as silicone, polytetrafluoroethylene (PTFE), or a hydrophilic coating. The lubricious interior surface of catheter 504 facilitates the longitudinal movement of delivery member 508 and deployment of restraining device 100.

In another embodiment of the invention, sheath 502 is retractable (not shown), as is well known in the art. Sheath 502 is retracted by the physician operator to deploy device 100 from delivery catheter 504. In this embodiment, delivery member 508 or a holding means may be used to maintain device 100 in a fixed position near catheter distal tip 506 until device 100 is deployed from the catheter.

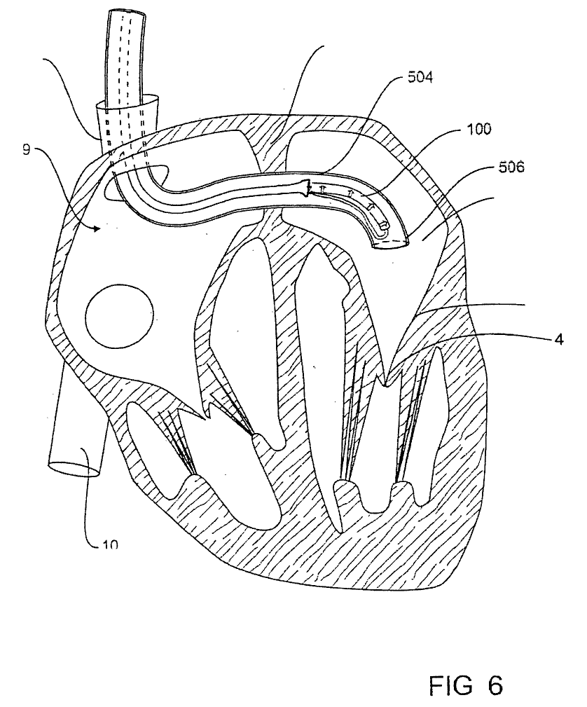

To deliver restraining device 100 adjacent to mitral valve 4 (FIG. 1), distal tip 506 of delivery catheter 504 containing device 100 is inserted into the vascular system of the patient. As shown in FIG. 6, catheter 504 may be inserted into the subclavian vein, through superior vena cava 8, and into right atrium 9. Alternatively, catheter tip 506 may be inserted through the femoral vein into the common iliac vein, through inferior vena cava 10, and into right atrium 9. Next, transeptal wall 11 between right atrium 9 and left atrium 12 is punctured with a guide wire or other puncturing device and distal tip 506 of delivery catheter 504 is advanced through the septal perforation and into left atrium 12 and placed in proximity to annulus 5 of mitral valve 4. Another possible delivery path would be through the femoral artery into the aorta, through the aortic valve into the left ventricle, and then through the mitral valve into left atrium 12. Yet another possible path would be through the left or right pulmonary vein directly into left atrium 12. The placement procedure, using any of these vascular routes, is preferably performed using fluoroscopic or echocardiographic guidance.

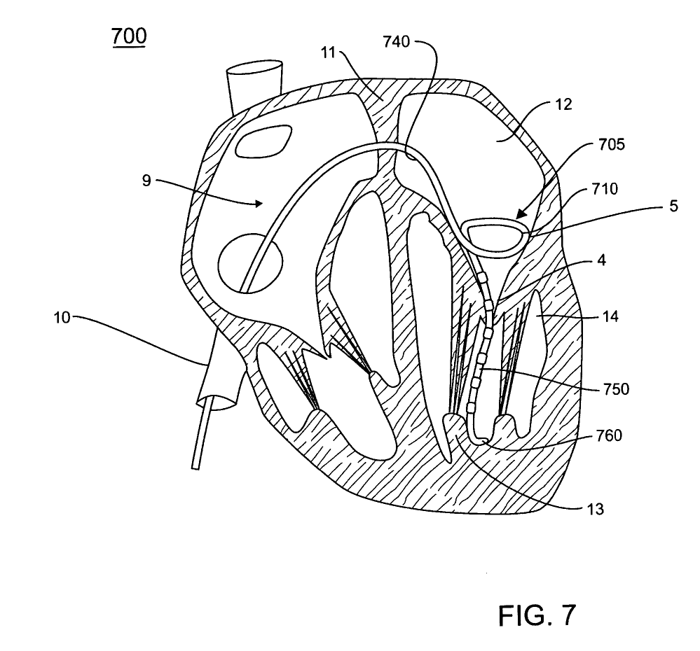

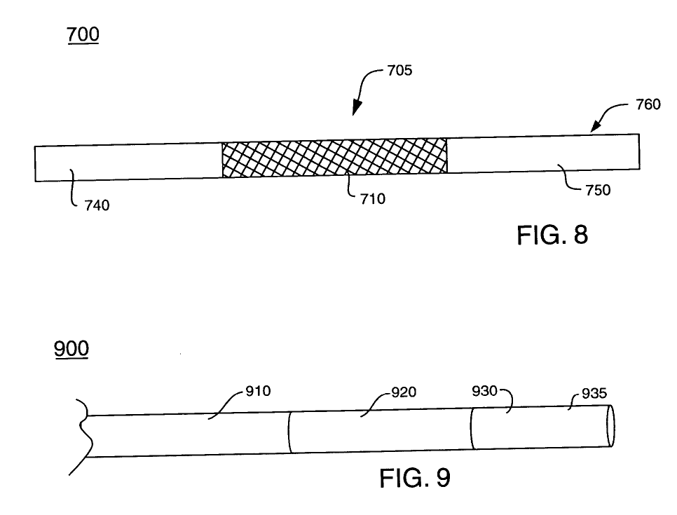

While the devices described herein can be delivered to a position adjacent a mitral valve annulus in a manner described above, other delivery systems and means can also be used. FIGS. 7 and 8 illustrate an embodiment of a delivery system 700 having a wireform that can be used as a guide for delivering at least one embodiment of the annulus restraining devices described herein. FIG. 7 illustrates an approach route in which catheters and/or guidewires are inserted into the femoral vein and passed through the common iliac vein, inferior vena cava 10, and into right atrium 9. Regardless of the route to right atrium 9, atrial septum 11 can be punctured with a guide wire or other puncturing device so that the annulus restraining device can be positioned in left atrium 12. In one embodiment of a delivery system for the devices, a puncture catheter, as is well known in the art, can be configured and used to pierce the wall of atrial septum 11. The delivery systems may also include a dilator catheter for providing a larger diameter pathway for delivering annulus reduction delivery system.

Referring to FIG. 7, delivery system 700 comprises wireform 705 having pre-shaped annular portion 710, proximal portion 740 and stabilizer portion 750. Wireform 705 may be composed of biocompatible metal, polymer or combinations thereof. In one embodiment, wireform 705 is pre-shaped and sized to fit the anatomy of a particular patient. In one embodiment, pre-shaped annular portion 710 comprises nitinol. In another embodiment, pre-shaped annular portion 710 comprises a section of tubular braid, either with or without a central monofilament core extending there through. Pre-shaped annular portion 710 provides a rail or guide for positioning an annulus reduction delivery system or device around and within the annulus 5 of mitral valve 4. FIG. 8 illustrates wireform 705 in a straight configuration as it may appear either during manufacture and before annular portion 710 is shaped, or as wireform 705 may temporarily appear during delivery to a cardiac valve through a delivery catheter.

Wireform stabilizer portion 750 extends distally from pre-shaped annular portion 710 and, in one embodiment, extends through the mitral valve 4 and into left ventricle 14. A stabilizer portion 750 traverses left ventricle 14 to rest on or near the apex of left ventricle 14 adjacent papillary muscles 13 to provide stability for wireform annular portion 710 during placement of an annulus restraining device. Stabilizer portion 750 may comprise a material that is relatively soft at distal tip 760 forming a pigtail or spiral shape as is known in the art. In another embodiment, stabilizing portion 750 extends from annular portion 710 in a superior direction to rest against an upper portion of left atrium 12 to provide stability. In another embodiment, wireform 705 does not include stabilizing portion 750. Delivery system 700 provides a pathway to and around mitral valve annulus 5 for delivering and positioning an annulus restraining device for implantation.

FIG. 9 illustrates delivery catheter 900 for delivering wireform 705 having a pre-shaped annular portion 710. Delivery catheter 900 includes proximal section 910, restraining section 920 and soft distal tip 930. Delivery catheter 900 comprises a flexible, biocompatible polymeric material such as polyurethane, polyethylene, nylon, or polytetrafluroethylene (PTFE). Additionally, restraining section 920 has sufficient stiffening capabilities to maintain pre-shaped annular portion 710 in a straightened delivery configuration. In one embodiment, a braided metallic or polymeric material is embedded in the wall of restraining section 920. In another embodiment metallic or polymeric rods are embedded in the wall of restraining section 920.

In operation, wireform 705 is inserted into delivery catheter 900. Delivery catheter 900 is then advanced to the target valve as described above. In one embodiment, distal end 935 is positioned within left atrium 12 and wireform 705 is pushed out of delivery catheter 900 to form delivery system 700 as seen in FIG. 7. In another embodiment, distal end 935 is advanced through mitral valve 4 and positioned adjacent papillary muscle 13. Delivery catheter 900 is then retracted while wireform 705 is held stationary. As delivery catheter 900 is retracted, delivery system 700 forms as seen in FIG. 7.

Once delivery system 700 is placed as seen in FIG. 7, delivery system 700 may be used to guide a suitable delivery catheter for annulus restraining device 100 to mitral valve annulus 5.

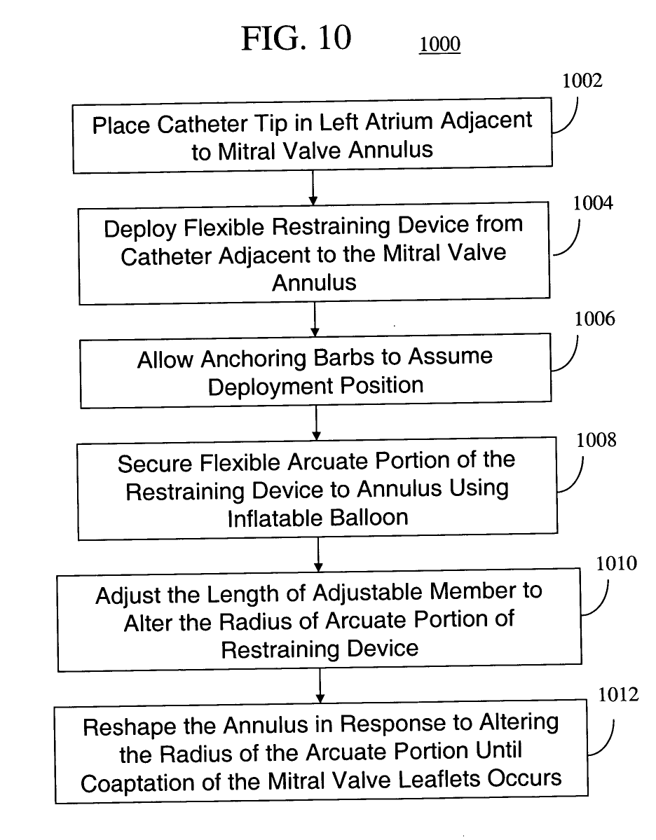

FIG.10 is a flowchart illustrating method 1000 for treating mitral valve regurgitation, in accordance with one aspect of the invention. As described in FIG.6, the distal tip of delivery catheter 504 containing flexible restraining device 100 is advanced through the vascular system of the patient, passed through right atrium 9 and into left atrium 12, adjacent to mitral valve annulus 5 (Block 1002). If a device such as delivery system 700 is used, wireform 705 is first delivered adjacent to the mitral valve of the patient using a delivery catheter such as catheter 900. As delivery system 700 is extruded from catheter 900, delivery system 700 takes the form seen in FIG. 7, and provides a guide for a delivery catheter suitable for annulus restraining device 100, such as delivery catheter 504.

Next, the restraining device is deployed adjacent to mitral valve annulus 5 from the delivery catheter (Block 1004). If a catheter such as catheter 504 is used, the flexible tip 506 is moved along the surface of mitral valve annulus 5, and used to direct the placement of restraining device 100. If delivery system 700 is used, the distal tip of a suitable catheter is guided along wireform 705. In either case, a deployment device, such as delivery member 508 within delivery catheter 504 is used to deploy restraining device 100 by pushing it from distal tip 506 of delivery catheter 504 and laying flexible restraining device 100 along mitral valve annulus 5. In yet another embodiment, sheath 502 is retracted to deploy restraining device 100.

Restraining device 100 is positioned so that anchor members 112 on the surface of restraining device 100 are facing the surface of mitral valve annulus 5. As restraining device 100 is extruded from distal tip 506 of delivery catheter 504, flexible member 102 of device 100 will assume a curved, nearly circular configuration commensurate with mitral valve annulus 5. In addition anchor members 112 assume a deployment configuration, in which they extend away from the surface of flexible member 102 at a predetermined angle (Block 1006).

In one embodiment of the invention, an inflatable balloon is then extended from distal tip 506 of delivery catheter 504 immediately adjacent to the surface of restraining device 100. The balloon may either be attached to distal portion 502 of delivery catheter 504, or it may be mounted on a separate catheter that is passed through delivery catheter 504. In either case, the balloon is inflated against restraining device 100 in order to push flexible member 102 against the surface of mitral valve annulus 5, with sufficient force to cause barbs 112 to penetrate mitral valve annulus 5, and to anchor restraining device 100 securely in place (Block 1008).

Once restraining device 100 is secured to mitral valve annulus 5 by anchor members 112, adjustment member 110 is manipulated so that the radius of flexible member 102 and the underlying mitral valve annulus are reduced by the desired amount (Block 1010). In one embodiment of the invention, flexible rod 508, used to deploy the restraining device 100 is withdrawn from the catheter, forceps are advanced through the catheter, and the tip of the forceps is placed adjacent to restraining device 100, which is attached to mitral valve annulus 5. Next, the forceps are used to grasp the adjustment elements 110, which in this embodiment are wires. Adjustment wires 110 are drawn together, and twisted around each other, causing the length of adjustment members 110 to be reduced, and ends 106 and 108 of flexible member 102 to be drawn toward each other, reducing the size of gap 104. In this embodiment, adjustment wires 110 remain twisted around each other, and maintain gap 104 at a fixed size. In another embodiment, a locking assembly, such as a clamp lock or any other appropriate device may be used to maintain the length of adjustment members 110. By drawing ends 106 and 108 of flexible member 102 together, the circumference of flexible member 102 is reduced, and, because restraining device 100 is securely fastened to annular ring 5 of mitral valve 4 (Block 1012), the circumference of annular ring 5 is reduced correspondingly. The circumference of mitral valve annular ring 5 is modified sufficiently so that anterior and posterior leaflets 7 and 6 close during ventricular contraction, and regurgitation of blood is reduced (Block 1012). Improvement in the valve closure can be evaluated by checking for decreased pressure in left atrium 12. Finally, delivery catheter 504 is withdrawn from the body of the patient.

While the invention has been described with reference to particular embodiments, it will be understood by one skilled in the art that variations and modifications may be made in form and detail without departing from the spirit and scope of the invention.

1. A system for treating mitral valve regurgitation, the system comprising:
a catheter;
a flexible restraining device having a plurality of anchor members extendable therefrom;
the anchor members being integrally formed with or fixedly attached to the flexible restraining device;
the anchor members shaped for penetration into the annulus of a mitral valve
the anchor members being movable from a delivery configuration to a deployment configuration; and
an adjustment member extending from end portions of the flexible restraining device.

2. The system of claim 1 wherein the flexible restraining device has an elongated, essentially linear configuration and an arcuate configuration;
the flexible restraining device has an inner surface and an outer surface and the anchor members are positionable in a catheter delivery configuration, in which the anchor members are parallel to the outer surface of the flexible restraining device; and
the anchor members are positionable in a deployment configuration, in which the anchor members extend radially from flexible restraining device and when the device is deployed from a catheter the anchor members extend to the deployment configuration.

3. The system of claim 1 wherein the flexible restraining device and anchor members comprise a shape memory alloy.

4. The system of claim 1 wherein the flexible restraining device and anchor members comprise nitinol.

5. The system of claim 1 further comprising means for seating the flexible restraining device against the annulus of a mitral valve and implanting the anchor members into the annulus.

6. The system of claim 1 wherein the catheter comprises:
an outer sheath;
a delivery chamber within the sheath at a distal end of the catheter; and
a deployment device positioned within the delivery chamber, wherein when the system is delivered to a location adjacent to a mitral valve, the flexible restraining device is deployed from the delivery chamber and positioned in a supra-annular position adjacent to the annulus of the mitral valve.

7. The system of claim 6 further wherein the catheter further comprises an inflatable balloon attached thereto and when the balloon is inflated, the flexible restraining device is seated against the annulus of the mitral valve.

8. The system of claim 1 wherein the adjustment member is selected from the group consisting of a string, a wire, a cord, a filament, and a cable.

9. The system of claim 8 wherein the flexible restraining device is a generally tubular member and the adjustment member is routed through the restraining device such that the ends of the adjustment member extend from the ends of the restraining device, and wherein when the restraining device is in an arcuate configuration, the adjustment member may be used to draw the ends of the flexible restraining device toward each other and maintain the ends of the restraining device in a fixed position.

10. A device for treating mitral valve regurgitation comprising:
a flexible restraining device having a plurality of anchor members extending therefrom;
the anchor members being integrally formed with or fixedly attached to the flexible restraining device;
the anchor members being movable from a delivery configuration to a deployment configuration; and
an adjustment member extending from end portions of the flexible restraining device.

11. The device of claim 10 wherein the flexible restraining device has an inner surface, an outer surface, an arcuate configuration and an elongated, essentially linear configuration;
the anchor members are positionable in a catheter delivery configuration, in which the anchor members lie on the outer surface of the flexible restraining device; and
the anchor members are positionable in a deployment configuration, in which the anchor members extend radially from flexible restraining device and when the device is deployed from a catheter the anchor members extend to the deployment configuration.

12. The device of claim 10 wherein the adjustment member is selected from the group consisting of a string, a wire, a cord, a filament, and a cable.

13. The device of claim 10 wherein the flexible restraining device is a generally tubular member and the adjustment member is routed through the restraining device such that the ends of the adjustment member extend from the ends of the restraining device, and wherein when the restraining device is in an arcuate configuration, the adjustment member may be used to draw the ends of the flexible restraining device toward each other and maintain the ends of the restraining device in a fixed position.

14. The device of claim 13 further comprising means for securing the ends of the adjustment members such the ends of the restraining device can be maintained in a fixed position.

15. The device of claim 10 wherein the flexible restraining device and anchor members comprise a shape memory alloy.

16. The system of claim 12 wherein the flexible restraining device and anchor members comprise nitinol.

17. A method of treating mitral valve regurgitation, the method comprising:
delivering a flexible restraining device, having shape-memory anchor members, to a location adjacent a mitral valve via a catheter;
deploying the flexible restraining device from the distal tip of the catheter thereby causing the flexible restraining device to assume an arcuate configuration;
moving the anchor members from a delivery to a deployment configuration responsive to the deployment of the flexible restraining device;
positioning the flexible restraining device against an annulus of the mitral valve;
inserting the anchor members into the annulus;
altering the shape of the flexible restraining device by altering the radius of the arcuate configuration; and
reshaping the annulus in response to the altering of the radius of the arcuate configuration the flexible restraining device.

18. The method of claim 17 wherein inserting the shape memory anchor members into the annulus further comprises inflating a balloon to exert force on the flexible ring, and causing the anchor members to penetrate the annulus.

19. The method of claim 17 wherein altering the radius of an arcuate configuration of the flexible restraining device further comprises drawing the ends of the device toward each other when the device is in the arcuate configuration.

20. The method of claim 17 wherein reshaping the annulus of the mitral valve reduces mitral valve regurgitation.
